(19) United States
(12) Patent Application Publication
(10) Pub. No.: US 2011/0028979 A1
(43) Pub. Date: Feb. 3, 2011

(54) Spinal Implant Clearing Gouge

(75) Inventor: Frank J. Schwab, New York, NY (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/533,536

(22) Filed: Jul. 31, 2009

(51) Int. Cl. A61B 17/00

(52) U.S. Cl. 606/83

(57) ABSTRACT

A surgical gouge for clearing bone and soft tissue from around a previously implanted rod during a surgical operation. The gouge includes an elongate handle with a distally positioned cutting member at one end. The cutting member has a rod capture arm with an arcuate surface generally conforming to the cylindrical shape of the rod. The cutting member further having a curved leading cutting blade extending over and about the rod capture arm. The curved leading cutting blade configured so as to create a trough between the inside surface of the curved leading cutting blade and the rod capture arm to capture any cut bone and/or soft tissue from around the rod as the gouge is rotated about the rod. The curved cutting blade having a distal end sharpened at its distal edge to enable the user to cut away bone and soft tissue from around the rod. It may also be desirable for the distal end to have sharpened side edges to assist in cutting away the bone and soft tissue that have built up about the rod.

### TECHNICAL FIELD

The present invention relates to a hand held surgical gouge for use in spinal implant revision surgery.

### BACKGROUND

The human spine is a biomechanical structure responsible for protecting the spinal cord, nerve roots and internal organs of the thorax and abdomen. The spine also provides structural support for the body while permitting flexibility of motion. In certain surgical procedures it is necessary to secure together two or more of the vertebral members. The procedure may be necessary for example as a result of physical trauma or degenerative disease.

One type of surgical procedure includes the attachment of a spinal rod to two or more vertebral members with pedicle screws. Such “rod assemblies” generally comprise one or two spinal rods and a plurality of pedicle screws inserted through the pedicles and into their respective vertebral bodies. Hooks can also be used to secure the rods to the vertebral bodies. The spinal rods extend along the longitudinal axis of the spine, coupling to the plurality of pedicle screws and/or hooks via their respective connectors. The aligning influence of the rods force the patient's spine to conform to a more appropriate shape.

In some instances, it becomes necessary or desirable to remove a rod that has been previously installed within a patient. This can take place years after the initial installation of the rod into the patient. During that time, significant bone or soft tissue growth can surround or cover the existing rod. It is desirable to have a ready hand-held surgical gouge that works to optimally remove the bone and soft tissue growth that captures and or covers the rod.

Additionally, it may be desirable to simply clear around the rod, versus actually removing the rod so that a new connector may be attached to the rod or to otherwise modify the spinal construct. Again, it would be beneficial to have a hand-held surgical gouge that works to readily clear new bone or soft tissue growth from around the previously implanted rod to allow for the installation of a new connector or other component onto the rod.\

Another type of surgical procedure includes the attachment of a plate to two or more vertebral members with bone screws. Such “plate assemblies” generally comprise a plate made of metal or other synthetic material fastened to the vertebrae with bone screws. These plate assemblies affix the two or more vertebral members together until bone fusion occurs there between. In some instances, it becomes necessary or desirable to remove a plate and bone screws that were previously implanted within a patient. In other instances, it may be desirable to simply clear any significant bone or soft tissue growth from about or around the plate assembly to otherwise modify the surgical implant. Again, it would be beneficial to have a hand-held surgical gouge that works to readily clear new bone or soft tissue growth from around the previously implanted plate assembly to allow the surgeon to properly modify the existing implant as needed.

### SUMMARY

The present invention is directed to an instrument for clearing bone and soft tissue from around a previously implanted implant during a surgical operation. The invention will be hereinafter described as clearing the bone and soft tissue from about a spinal rod; however the surgical gouge of the present invention is equally capable of clearing such bone and tissue from about any surgical rod, or other vertebral longitudinal member, or plate assembly, or other implant that may be placed within the human body. The rod is typically made from stainless steel or titanium, however may also be constructed of other material such as a hard plastic (i.e., PEEK) or other synthetic type material.

In one embodiment of a spinal rod clearing gouge for use in clearing bone and soft tissue form about a spinal rod, the gouge includes an elongate handle with a distally positioned cutting member at one end. The cutting member has a rod capture arm with an arcuate surface generally conforming to the cylindrical shape of the rod. The arcuate surface having sharp edges on both ends of the arcuate surface partially surrounding the rod when the gouge is in position adjacent the rod to be cleared. It may be desirable for the arcuate edges of the arcuate surface to be sharpened to allow such edges to cut bone and soft tissue away from about the rod or touching the rod.

The cutting member further having a curved leading cutting blade extending over and about the rod capture arm. The curved leading cutting blade configured so as to create a trough between the inside surface of the curved leading cutting blade and the rod capture arm to capture any cut bone and/or soft tissue from around the rod as the gouge is rotated about the rod. The curved cutting blade having a distal end sharpened at its distal edge to enable the user to cut away bone and soft tissue from around the rod. It may also be desirable for the distal end to have sharpened side edges to assist in cutting away the bone and soft tissue that have built up about the rod.

It may also be desirable for the elongate handle of the rod clearing gouge to have a proximal end that has an enlarged knob suitable for being struck by a hammer or mallet to assist the distal cutting member in clearing the bone or soft tissue from about the rod.

In another embodiment, the elongate handle has an enlarged portion a spaced distance from its proximal end. A cylindrical weight is positioned about the elongate handle proximally from the enlarged portion. The cylindrical weight may be a separate part from the gouge or may be captured about the elongate handle between the enlarged portion and the proximal end. The weight is used to apply an axial force along the elongate handle in a distal direction to assist the distal cutting member in removing bone and/or soft tissue from about the rod.

In another embodiment, the distally positioned cutting member may be detachable from the elongate handle to allow for the cutting member to be cleaned and/or sharpened. A plurality of cutting members may be provided having different configurations to allow for the provision of different rod capture arms having different arcuate surfaces for mating with different diameter rods. The use of different cutting members would also allow for the provision of different blade configurations and shapes to allow for easy removal of bone and/or soft tissue from about plate assemblies or other surgical implants positioned within the body. The cutting blade widths may be varied or the radius of the curved leading cutting blade may also be varied as is desired by the surgeon.

The present invention further provides a method of removing bone and soft tissue from about a previously installed rod in the human body. The method includes providing a gouge instrument for clearing bone and soft tissue from about a rod in accordance with any of the embodiments described herein. One method includes positioning the gouge next to a previously installed rod that has significant boney or soft tissue ingrowth around the rod. The surgeon places the rod capture arm of the gouge adjacent the rod with the curved leading cutting blade adjacent the bone or soft tissue. The surgeon then rotates the gouge into the bone or soft tissue such that the gouge rotates about the rod with the cutting blade cutting into the bone or soft tissue that has grown about the rod. The rod capture arm is in contact with and rotates about the rod which acts as a bearing surface allowing the surgeon to use hand force to move the cutting blade into the bone or soft tissue. The cut bone and soft tissue will collect in the tough of the gouge to allow the surgeon to remove such tissue when lifting the gouge off of the rod. The surgeon can continue cutting away the tissue in the same direction or can reverse the gouge to rest the rod capture arm of the gouge on the rod and push the cutting blade into the bone or soft tissue on the opposite side of the rod. Furthermore, the sharp surfaces on the edges of the rod capture arm will also cut the tissue away from the rod when the gouge is rotated about the rod in either direction. The surgeon may also want to use a hammer or mallet to lightly strike the proximal end of the gouge to assist in cutting the bone or soft tissue away from the rod.

An alternate method of removing bone and soft tissue from about a previously installed rod in the human body, is to use the gouge of the present invention having a weight positioned about the proximal end of the elongated handle and to move the weight against the integral enlarged portion to force the curved leading cutting blade into the bone or soft tissue surrounding the rod. The integral weight works like a slap hammer to force the cutting blade into the tissue to assist in the removal thereof.

Other systems, methods, features and advantages of the invention will be, or will become apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 is an isometric view of a gouge for clearing bone or soft tissue from about a previously installed rod within the human body;

FIG. 2 shows a cross sectional view of a spinal rod with bone or soft tissue growing up and around the sides of the rod;

FIG. 3 shows a cross sectional view of the rod with bone or soft tissue growth about the rod as shown in FIG. 2 with the gouge of the present invention adjacent the rod with the gouge being rotated in a clockwise direction about the rod cutting the bone and soft tissue away from the rod;

FIG. 4 shows the same view as FIG. 3 with the gouge being rotated in a counter clockwise direction about the rod to remove the bone and soft tissue from about the rod;

FIG. 5 shows an isometric view of an another embodiment of the bone clearing gouge of the present invention; and

FIG. 6 shows an isometric view of yet another embodiment of the bone clearing gouge of the present invention.

### DETAILED DESCRIPTION

For the purposes of promoting an understanding of the principles of the present invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

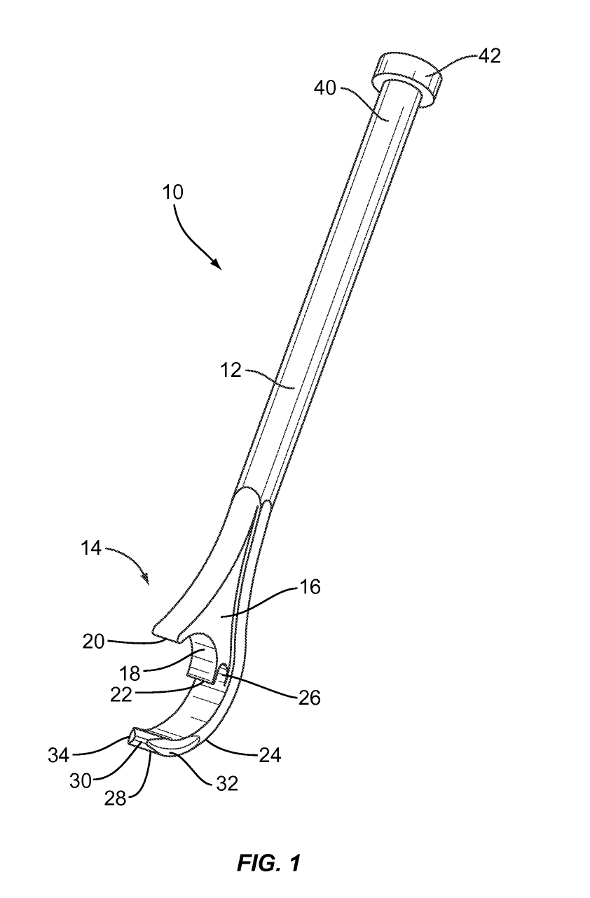

The present invention is directed to an instrument for clearing bone or soft tissue from around a previously implanted implant during a surgical operation. As will be described below, the implant described herein is a spinal rod which would have been implanted during a spinal operation. In some instances, a surgeon would want to later either remove the rod or simply clear around the rod to add a connector to the rod to enlarge the original spinal construct or otherwise modify the original spinal construct. FIG. 1 illustrates one embodiment of the bone and/or soft tissue clearing gouge 10 including a proximally positioned elongate handle 12 at one end and a distally positioned cutting member 14 at the other end. For ease of description, the directions distal and proximal shall refer to the ends of the gouge that are either farther away from or closer to the hand of the surgeon user when grasping the gouge, respectively. The cutting member 14 has a rod capture arm 16 with an arcuate surface 18 generally conforming to the cylindrical shape of the rod (R). The arcuate surface 18 has edges 20 and 22 which also may be sharpened to assist in cutting the bone or soft tissue away from about the rod.

Still referring to FIG. 1, the cutting member 14 has a curved leading cutting blade 24 extending over and about the rod capture arm 16. The curved leading cutting blade 24 is configured so as to create a trough 26 between the inside surface of the cutting blade 24 and the rod capture arm 16. The trough 26 is meant to collect and/or capture any cut bone or soft tissue so that such material can be easily removed from the surgical site. The curved cutting blade 24 has a distal end 28 which may have a sharpened edge 30 to enable the surgeon user to cut away bone and soft tissue that has grown up around the implanted rod as more fully discussed below. It may also be desirable for the distal end 28 to have sharpened side edges 32 and 34 to further assist in cutting away the bone and soft tissue from about the rod. It may also be desirable for the cutting member 14 to be removable from the elongate handle 12 to allow the cutting member 14 to be cleaned and/or sharpened. Furthermore, a plurality of cutting members 14 may then be provided having different rod capture arm radius surfaces to match different rod diameters, or different curved leading cutting blade 24 configurations to allow the gouge to be used in different applications to remove any bone and/or soft tissue build up around various spinal or surgical implants within the body.

It may also be desirable for the elongate handle 12 of the rod clearing gouge 10 to have a proximal end 40 that has an enlarged knob 42 suitable for being struck by a hammer or mallet to assist the distal cutting member 14 in clearing the bone and soft tissue from about the implanted rod.

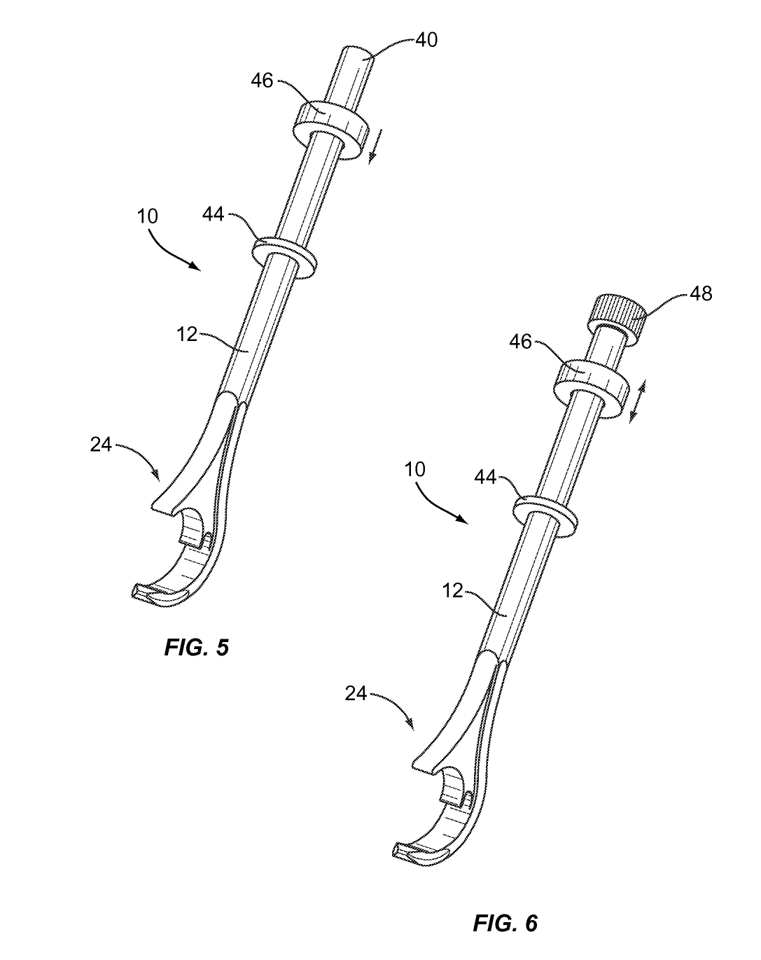

Referring to FIG. 5, another embodiment of the rod clearing gouge 10 is shown. In this embodiment, the elongate handle 12 has an enlarged portion 44 a spaced distance from its proximal end 40. A cylindrical weight 46 can be positioned about the elongate handle 12 at its proximal end 40 to apply an axial force along the gouge to assist the user in cutting any bone and soft tissue from about the rod. And, referring to FIG. 6, another embodiment is shown wherein the weight 46 is captured around the elongate handle 12 between the enlarged portion 44 and a removable knob end 48.

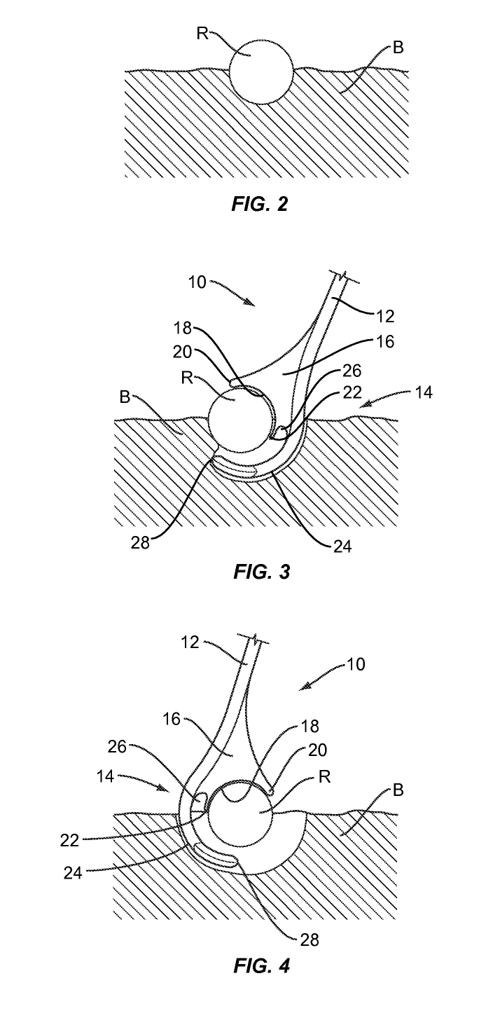

Referring to FIGS. 2 thru 4, the method of removing bone and soft tissue form about a previously installed rod using the rod clearing gouge 10 of the present invention is more clearly shown. FIG. 2 depicts a rod (R) which has been implanted within a human body where bone and soft tissue (B) has grown up around the sides of the rod. In some cases the bone and soft tissue may totally cover the implanted rod. To use the rod clearing gouge 10 of the present invention, the surgeon user need only clear the uppermost portion or top of the rod so that the rod surface can be visualized.

Referring to FIG. 3, the surgeon user places the rod capture arm 16 on top of the rod and pushes the curved leading cutting blade 24 into the bone and soft tissue that needs to be removed. In FIG. 3, the user is rotating the cutting blade in a clockwise direction. The directions clockwise and counter clockwise are used for convenience and only in reference to FIGS. 3 and 4. The distal end 28 and sharpened edge 30 will dig an arcuate path into the tissue to be removed as the arcuate surface 18 of the rod capture arm 16 follows the cylindrical surface of the rod (R). The sharpened edges 20 and 22 of the rod capture arm 16 would also cut away bone and soft tissue from the rod while rotating the gouge about the rod. The cut bone and soft tissue will collect in the tough 26 of the gouge 10 so that the surgeon user can remove such tissue from the surgical site. The surgeon user would then replace the rod capture arm 16 of the gouge 10 back on the rod and continue cutting away any necessary bone and soft tissue from about the rod in the same clockwise direction until all such material is removed from that side of the rod. The surgeon user may want to use a hammer or mallet to lightly strike the enlarged knob 42 on the proximal end 40 of the gouge 10 to assist in cutting the bone and soft tissue from about the rod.

Referring to FIG. 4, the surgeon user can now place the arcuate surface 18 of rod capture arm 16 of the gouge 10 on the rod (R) with the cutting blade 24 on the other side of the rod and rotate the cutting member 14 in a counter clockwise direction to remove bone and soft tissue from about the opposite side of the rod. As shown in FIG. 4, in this fashion the surgeon user can remove all of the bone and/or soft tissue from around a rod to leave it totally clear to attach a connector to the rod or otherwise modify the spinal construct. Again, the surgeon user may want to use a hammer or mallet to lightly strike the enlarged knob 42 of the proximal end 40 of the gouge to assist in cutting the bone and soft tissue from about the rod.

Furthermore, referring to both FIGS. 3 and 4, the surgeon user can also use the sharpened side edges 32 and 34 of the curved leading cutting blade 24 to cut axially along the rod. The surgeon user would rest the arcuate surface 18 of the rod capture arm 16 on the rod and move the gouge 10 axially along the rod to cut bone and soft tissue away from the rod.

An alternate method of removing bone and soft tissue from about a previous implanted rod, is to use the gouge of FIG. 5, having a weight 46 positioned about the proximal end 40 of the elongate handle 12 and to move the weight against an integrally enlarged portion 44 of the handle 12 to apply axial force along the gouge to move the curved leading cutting blade 24 into the bone and soft tissue to be removed. The surgeon user would apply the axial force of the weight 46 along the elongate handle 12 with a rotational twist of the user's hand to force the curved cutting blade 24 into the tissue to be removed.

The method could also use the gouge 10 of FIG. 6, wherein the weight 46 is captured along the elongate handle 12 between an enlarged portion 44 and a removable knob 48. The same force and motion as discussed in the previous paragraph would be used to cut away the bone and soft tissue from about the implanted rod.

While the invention has been illustrated and described in detail in the drawings and foregoing description, the same is to be considered as illustrative and not restrictive in character.

What is claimed is:

1. A gouge for clearing bone and soft tissue from about a rod which has been previously implanted within a human being, the gouge comprising:
an elongate handle at a proximal end;
a cutting member at a distal end; and
the cutting member having a rod capture arm having an arcuate surface generally conforming to the cylindrical shape of the rod, and a curved leading cutting blade extending over the rod capture arm;
such that when the rod capture arm is placed in contact with the rod and rotated about such rod toward the curved leading cutting blade the cutting blade will cut away any bone and soft tissue adjacent the rod.

2. The gouge of claim 1, wherein a trough is created between the inside surface of said curved leading cutting blade and said rod capture arm to collect any bone and soft tissue cut from about the rod.

3. The gouge of claim 2, wherein said curved leading cutting blade further comprises a leading cutting edge and sharpened side edges to allow said gouge to cut away bone and soft tissue from the rod in both radial and axial directions as the gouge is rotated about and along the rod.

4. The gouge of claim 1, wherein said arcuate surface of said rod capture arm has sharp edges on both ends of the arcuate surface to assist in cutting bone and soft tissue away from the rod as the gouge is rotated about the rod.

5. The gouge of claim 1, wherein said elongate handle has an enlarged proximal end suitable for being struck by a hammer to assist the cutting member from removing bone and soft tissue from about the rod.

6. The gouge of claim 1, wherein said elongate handle has an integral enlarged portion and said handle can receive a cylindrical weight about it proximal end, the weight can be moved axially of the handle to strike said enlarged portion to assist said cutting member in cutting bone and soft tissue away from about the rod.

7. The gouge of claim 6, wherein the weight is captured about the elongate handle between the integral enlarged portion and a removable knob at its proximal end.

8. The gouge of claim 3, wherein the cutting member is removable from the elongate handle such a plurality of cutting members can be used with said elongate handle to provide various cutting members having different configurations and shapes.

9. A gouge for clearing bone and soft tissue from about a rod which has been previously implanted within a human being, the gouge comprising:
an elongate handle at a proximal end;
a cutting member at a distal end;
the cutting member having a rod capture arm having an arcuate surface generally conforming to the cylindrical shape of the rod, and a curved leading cutting blade extending over the rod capture arm, the curved leading cutting blade having an inside surface facing said rod capture arm; and
a trough being created between said inside surface of the curved leading cutting blade and the rod capture arm;
such that when the rod capture arm is placed in contact with the rod and rotated about such rod toward the curved leading cutting blade the cutting blade will cut away any bone and soft tissue adjacent the rod, such bone and soft tissue being captured within said trough for easy removal from the surgical site.

10. The gouge of claim 9, wherein said curved leading cutting blade further comprises a leading cutting edge and sharpened side edges to allow said gouge to cut away bone and soft tissue from the rod in both radial and axial directions as the gouge is rotated about and along the rod.

11. The gouge of claim 9, wherein said arcuate surface of said rod capture arm has sharp edges on both ends of the arcuate surface to assist in cutting bone and soft tissue away from the rod as the gouge is rotated about the rod.

12. The gouge of claim 9, wherein said elongate handle has an enlarged proximal end suitable for being struck by a hammer to assist the cutting member from removing bone and soft tissue from about the rod.

13. The gouge of claim 9, wherein said elongate handle has an integral enlarged portion and said handle can receive a cylindrical weight about it proximal end, the weight can be moved axially of the handle to strike said enlarged portion to assist said cutting member in cutting bone and soft tissue away from about the rod.

14. The gouge of claim 13, wherein the weight is captured about the elongate handle between the integral enlarged portion and a removable knob at its proximal end.

15. The gouge of claim 11, wherein the cutting member is removable from the elongate handle such a plurality of cutting members can be used with said elongate handle to provide various cutting members having different configurations and shapes.

16. A method of removing bone and soft tissue from about an implanted rod within a human body, comprising:
providing a gouge having an elongate handle and a cutting member at a distal end, the cutting member having a rod capture arm having an arcuate surface generally conforming to the cylindrical shape of the rod, and a curved leading cutting blade extending over the rod capture arm; and
placing the arcuate surface of the rod capture arm onto the rod with the distal end of the curved leading cutting blade adjacent the bone and soft tissue to be removed from about the rod; and
rotating the gouge in a direction towards the distal end of the curved leading cutting blade with the arcuate surface of the rod capture arm bearing on the rod so that the curved leading cutting blade will cut an arcuate path into the bone and soft tissue surrounding the rod.

17. The method of claim 16, wherein said gouge further comprises a trough provided between the inside surface of the curved leading cutting blade and the rod capture arm; and
collecting any bone and soft tissue cut by the curved leading cutting blade in said trough for removal from the surgical site.

18. The method of claim 16, wherein after removal of any bone and soft tissue from said trough to replace the gouge back onto the rod to further cut bone and soft tissue from about the rod until all of such tissue has been removed from about the rod.
